(19) United States
(12) Patent Application Publication
(10) Pub. No.: US 2019/0250154 A1
(43) Pub. Date: Aug. 15, 2019

(54) METHOD FOR RE-USING TEST PROBE AND REAGENTS IN AN IMMUNOASSAY BASED ON INTERFEROMETRY

(71) Applicant: Access Medical Systems, Ltd., Palo Alto, CA (US)

(72) Inventors: Robert F. Zuk, Menlo Park, CA (US); Qing Xia, Sunnyvale, CA (US)

(21) Appl. No.: 16/394,953

(22) Filed: Apr. 25, 2019

Related U.S. Application Data
(63) Continuation of application No. PCT/US2017/058878, filed on Oct. 27, 2017.
(60) Provisional application No. 62/415,247, filed on Oct. 31, 2016.

(51) Int. Cl. G01N 33/543

(52) U.S. Cl. CPC G01N 33/543

(57) ABSTRACT

The present invention is directed an immunoassay method using an interference detection system. The assay re-uses an antibody-immobilized test probe and reagents for quantitating an analyte in different samples, from about 3 to 20 times, while maintaining acceptable clinical assay performance. The method regenerates the test probe with an acidic solution after completion of each cycle of reactions. The present invention is also directed to a unitized cartridge (a strip) for an immunoassay test. Each unitized cartridge contains all necessary reagents and can be used for 3-20 cycles to measure 3-20 different samples.

This application is a continuation of PCT/US2017/058878, filed Oct. 27, 2017; which claims the benefit of U.S. Provisional Application No. 62/415,247, filed Oct. 31, 2016. The contents of the above-identified applications are incorporated herein by reference in their entirety.

### TECHNICAL FIELD

The present invention relates to a method to re-use an immunoassay test probe from about 3 to 20 times, in a thin film interferometry detection system.

### BACKGROUND

Cost containment is a major goal for healthcare providers worldwide. In vitro diagnostics (IVD) is no exception, where the clinical utility of biomarkers in the diagnosis and prognosis has become standard in patient management. Immunoassay technology is large portion of the WD industry and is steadily growing, about 3%/year in the U.S. and 15-20%/year in developing countries. In some cases, such as serial measurements for cardiac markers in diagnosing myocardial infarction, cost can limit the appropriate amount of testing.

### BRIEF DESCRIPTION OF THE DRAWINGS

FIG. 1 shows a bio-sensor interferometer including a lens.

FIG. 2 illustrates a typical interference pattern of a binding assay detected by a thin-film interferometer.

FIG. 3A illustrates bio-sensor interferometer including a coupling hub. FIG. 3B shows a probe inserted into a coupling hub.

FIG. 4 illustrates a first assay protocol. Ab=antibody. Sample contains an antigen analyte.

FIG. 5 illustrates a second assay (sandwich format) protocol, in which C-reactive protein (CRP) is an analyte. The probe is immobilized with a first anti-CRP antibody, and the reagent vessel contains a second anti-CRP antibody.

FIG. 6A shows the wavelength phase shift (nm) of Cycle 1 to Cycle 10 when anti-CRP antibody CRP 30 was used as a capture antibody. The results show consistent wavelength phase shifts from Cycle 1 to Cycle 10. FIG. 6B shows the wavelength phase shift (nm) of Cycle 1 to Cycle 9 when anti-CRP antibody C7 was used as a capture antibody. The results show that wavelength phase shift dropped significantly from Cycle 1 to Cycle 9.

FIG. 7 shows the results of wavelength phase shift (nm) of Cycle 1 to Cycle 10 when anti-CRP antibody CRP 30 was used as a capture antibody, and anti-CRP antibody C5 was used as a signal antibody. The results show consistent wavelength phase shifts from Cycle 1 to Cycle 10.

### DETAILED DESCRIPTION OF THE INVENTION

### Definitions

Terms used in the claims and specification are to be construed in accordance with their usual meaning as understood by one skilled in the art except and as defined as set forth below.

“About,” as used herein, refers to within ±10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding partner for that protein; (iv) ligands, for use in detecting the presence of a binding partner; or (v) single stranded nucleic acid molecules, for detecting the presence of nucleic acid binding molecules.

An “aspect ratio” of a shape refers to the ratio of its longer dimension to its shorter dimension.

A “binding molecular,” refers to a molecule that is capable to bind another molecule of interest.

A “ferrule” as used herein, refers to a rigid tube that confines or holds a waveguide as part of a connector assembly.

“Immobilized,” as used herein, refers to reagents being fixed to a solid surface. When a reagent is immobilized to a solid surface, it is either be non-covalently bound or covalently bound to the surface.

“A monolithic substrate,” as used herein, refers to a single piece of a solid material such as glass, quartz, or plastic that has one refractive index.

A “probe,” as used herein, refers to a substrate coated with a thin-film layer of analyte-binding molecules at the sensing side. A probe has a distal end and a proximal end. The proximal end (also refers to probe tip in the application) has a sensing surface coated with a thin layer of analyte-binding molecules.

A “waveguide” as used herein, refers to a device (as a duct, coaxial cable, or optic fiber) designed to confine and direct the propagation of electromagnetic waves (as light); for example, a waveguide is a metal tube for channeling ultrahigh-frequency waves.

A “waveguide connector” as used herein, refers to a mechanical device for optically joining the locking together separable mating parts of a waveguide system. It is also known as a waveguide coupler.

The present invention discloses a method of re-using an immunoassay test probe and reagents in an immunoassay based on interferometry, from about 3 to 20 times, while maintaining acceptable clinical assay performance. The immunoassay test probe and reagents may be contained in one test strip, or one cartridge. The present invention re-uses test probe and reagents and saves the cost on a per test basis.

There are several key elements to practice the invention. First, the invention regenerates the test probe by employing a low pH denaturing reagent that disassociates the immune complexes bound to the antibodies immobilized on a solid phase, but does not denature or disassociate the antibodies bound to the solid phase to a degree that affects the assay performance. The denaturation step conditions the solid phase antibody for subsequent binding steps to other antigen containing samples. Second, the probe tip has a small dimension (≤5 mm in diameter) so that there is negligible consumption of the reagents, and no replenish of the reagents is necessary during the assay cycles. Third, the assay utilizes the same test probe and the same reagents necessary to perform a complete assay, which facilitates multiple assay cycles without additional reagents.

### Interference Detection System

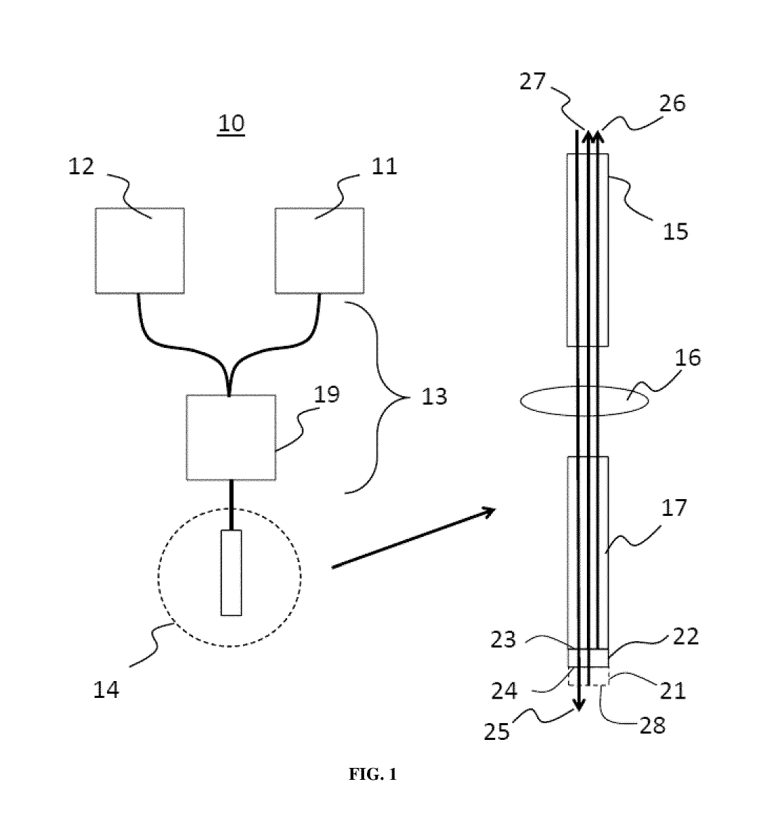

In one embodiment, the present invention uses an interference detection system as shown in FIG. 1. FIG. 1 illustrates an example of a biosensor interferometer 10 including a lens 16. A biosensor interferometer 10 comprises a light source 11, a detector 12, a waveguide coupler 19, waveguide 13 and an optical assembly 14. The optical assembly 14 comprises the tip of the waveguide (also referred to as waveguide tip) 15, a lens 16, a monolithic substrate 17, a thin-film layer (interference layer) 22 and a biomolecular layer 21. The thin-film layer 22 can include a transparent material. The thin-film layer 22 has a sensing surface 24 and a reflecting surface 23. The layer of biomolecular molecules 21 is attached to the thin-film layer 22 at the surface 24. The reflecting surface 23 is between the thin-film layer 22 and the monolithic substrate 17. The surface 24 between the thin-film layer 22 and the biomolecular layer 21 is also referred to as a “sensing surface.”

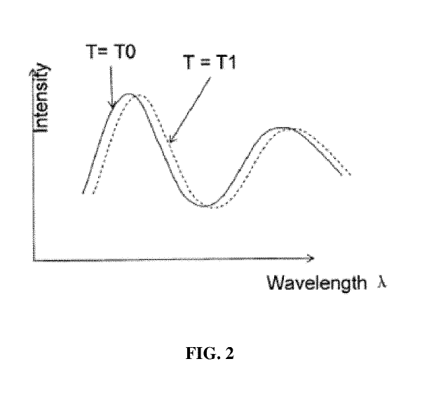

The two light signals 26, 27 reflected from boundaries between the first and second reflecting surfaces 23, 28 generate a spectral interference pattern, as shown in FIG. 2. When biomolecules bind to analyte molecules on the peripheral surface of the thin-film layer 22 to form an interference layer, the equivalent optical path of the second reflection signal 27 extends. As a result, the spectral interference pattern shifts from T0 to T1 as shown in FIG. 2. By measuring the pattern's phase shift continuously in real time, a kinetic binding curve can be measured as the amount of shift vs. the time. The association rate of an analyte to a capture molecule immobilized on the surface can be used to calculate the analyte's concentration. Hence, the measurement of this phase shift is the detection principle of a thin-film interferometer.

In one embodiment, the present invention uses an interference detection system as described in U.S. Pat. No. 8,597,578, which is incorporated herein by reference in its entirety, for measuring the interference pattern on a probe tip.

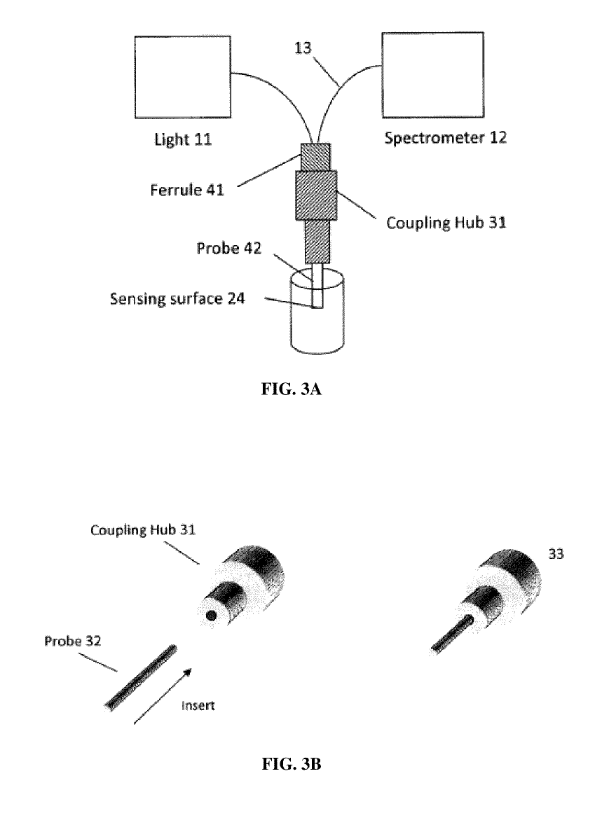

In one embodiment, the interference detection system comprises a coupling hub as illustrated in FIG. 3A. FIG. 3A shows a simplified illustration of the bio-sensor based on thin film interferometer. The bio-sensor comprises light source 11, spectrometer 12, waveguide 13, ferrule 41, coupling hub 31, probe 42, and sensing surface 24. The tip of the probe and the sensing surface 24 are dipped into a coating solution containing analyte-binding molecules. FIG. 3B shows a probe 32 is inserted into the center bores of the molded plastic 31 and results a structure 33.

In other embodiments, the interference detection systems described in the following U.S. Pat. Nos. 5,804,453, 7,394,547, and 7,319,525 can be used in the present method.

U.S. Pat. No. 5,804,453 discloses a method of determining the concentration of a substance in a sample solution, using a fiber optic having a reagent (capturing molecule) coated directly at its distal end to which the substance binds. The distal end is then immersed into the sample containing the analyte. Binding of the analyte to the reagent layer generates an interference pattern and is detected by a spectrometer. The interference detection systems described in this patent is incorporated herein by reference.

U.S. Pat. No. 7,394,547 discloses a biosensor that a first optically transparent element is mechanical attached to an optic fiber tip with an air gap between them, and a second optical element as the interference layer with a thickness greater than 50 nm is then attached to the distal end of the first element. The biolayer is formed on the peripheral surface of the second optical element. An additional reflective surface layer with a thickness between 5-50 nm and a refractive index greater than 1.8 is coated between the interference layer and the first element. The interference detection systems described in this patent is incorporated herein by reference.

U.S. Pat. No. 7,319,525 discloses a different configuration that a section of an optic fiber is mechanically attached to a tip connector consisting of one or more optic fibers with an air gap between the proximal end of the optic fiber section and the tip connector. The interference layer and then the biolayer are built on the distal surface of the optical fiber section. The interference detection systems described in this patent is incorporated herein by reference.

An interference layer (a thin-film layer) is a transparent material coated on the sensing side of the monolithic substrate. Thin films are thin material layers ranging from fractions of a nanometer (monolayer) to several micrometers in thickness. The thin-film layer of the present invention typically has a thickness of at least 50 nm, and preferably at least 100 nm. An exemplary thickness is between about 100-5,000 nm, preferably 400-1,000 nm. The refractive index of the thin-film layer material is preferably similar to that of the first reflecting surface, so that reflection from the lower distal end of the optical assembly occurs predominantly from the layer formed by the analyte-binding molecules, rather than from the interface between the optical element and the analyte-binding molecules. Similarly, as analyte molecules bind to the lower layer of the optical assembly, light reflection form the lower end of the assembly occurs predominantly from the layer formed by the analyte-binding molecules and bound analyte, rather than from the interface region. One exemplary material forming the thin-film layer is SiO₂. The thin-film layer can also be formed of a transparent polymer as the monolithic substrate, such as polystyrene or polyethylene.

The thickness of the biomolecular (analyte-binding molecular) layer is designed to optimize the overall sensitivity based on specific hardware and optical components. Conventional immobilization chemistries are used in chemically, e.g., covalently, attaching a layer of analyte-binding molecules to the lower surface of the optical element. For example, a variety of bifunctional reagents containing a siloxane group for chemical attachment to SiO₂, and an hydroxyl, amine, carboxyl or other reaction group for attachment of biological molecules, such as proteins (e.g., antigens, antibodies), or nucleic acids. It is also well known to etch or otherwise treat glass a glass surface to increase the density of hydroxyl groups by which analyte-binding molecules can be bound. When the thin-film layer is formed of a polymer, such as polystyrene, a variety of methods are available for exposing available chemically-active surface groups, such as amine, hydroxyl, and carboxyl groups.

The analyte-binding layer is preferably formed under conditions in which the distal surface of the optical element is densely coated, so that binding of analyte molecules to the layer forces a change in the thickness of the layer, rather than filling in the layer. The analyte-binding layer can be either a monolayer or a multi-layer matrix.

The measurement of the presence, concentration, and/or binding rate of analyte to the optical assembly is performed by the interference of reflected light beams from the two reflecting surfaces in the optical assembly. Specifically, as analyte molecules attach to or detach from the surface, the average thickness of the first reflecting layer changes accordingly. Because the thickness of all other layers remains the same, the interference wave formed by the light waves reflected from the two surfaces is phase shifted in accordance with the thickness change due to the analyte binding.

### The Probe

The probe can be a monolithic substrate or an optical fiber. The probe can be any shape such as rod, cylindrical, round, square, triangle, etc., with an aspect ratio of length to width of at least 5 to 1, preferably 10 to 1. Because the probe is dipped in a sample solution and one or more reagent solutions during an immunoassay, it is desirable to have a long probe with an aspect ratio of at least 5 to 1 to enable the probe tip's immersion into the solutions. Heterogeneous assays can be performed where the long probe is transferred to different reaction chambers. Dispensing and aspirating reagents and sample during the assay are avoided. In one preferred embodiment, the diameter of the probe tip surface is ≤5 mm, or ≤2 mm. The sensing surface of the probe tip is coated with analyte-binding molecules.

Methods to immobilize a first antibody to the solid phase (the sensing surface of the probe tip) are common in immunochemistry and involve formation of covalent, hydrophobic or electrostatic bonds between the solid phase and antibody. The first antibody, also called capture antibody for its ability to capture the analyte, can be directly immobilized on the sensing surface. For example, a first antibody can be first immobilized either by adsorption to the solid surface or by covalently binding to aminopropylsilane coated on the solid surface. Alternatively, the first antibody can be indirectly immobilized on the sensing surface through a binding pair. For example, the first antibody can be labeled with biotin by known techniques (see Wilchek and Bayer, (1988) Anal. Biochem. 171:1-32), and then be indirectly immobilized on the sensing surface coated with streptavidin. Biotin and streptavidin are a preferred binding pair due to their strong binding affinity, which does not dissociate during the low pH (pH 1-4) regeneration steps of the present method. The capture antibody immobilized on the sensing surface must be able to survive the denaturation condition when the probe sensing surface is regenerated to remove the immunocomplex bound to the sensing surface after the immunoreaction. The capture antibody immobilized on the sensing surface must not lose a significant amount of activity or significantly disassociate from the solid phase so that the immunoassay performance is compromised.

### Detecting an Analyte by a Recycling Protocol—First Embodiment

The present invention is directed to a method of detecting an analyte in multiple liquid samples by an immunoassay in a interferometry detection system, using the same test probe and same test reagents for different sample.

In a first embodiment, a capture antibody is immobilized on the probe, and the sample contains an antigen analyte.

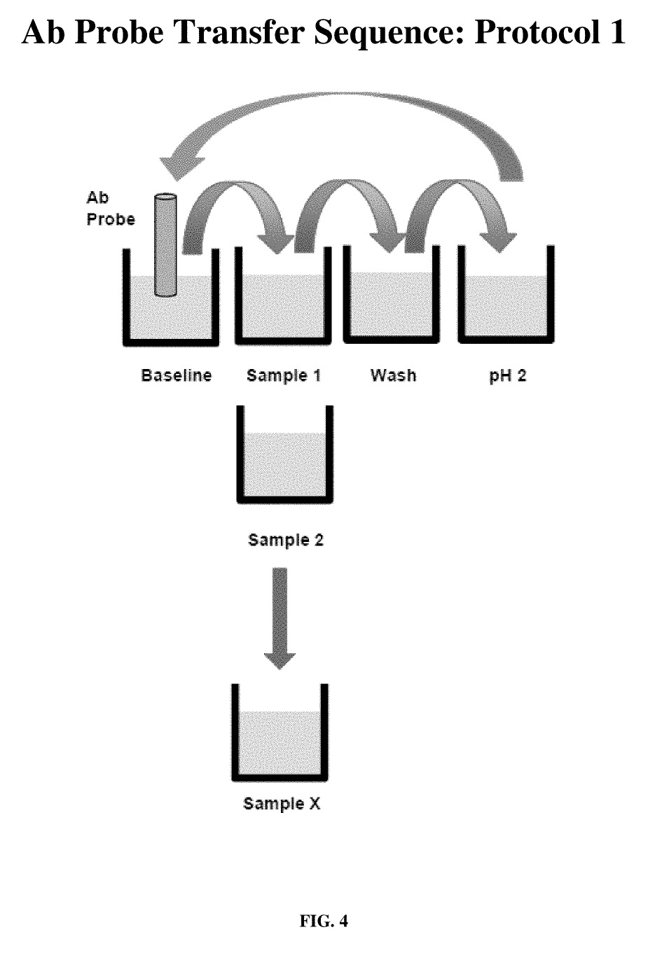

The first method comprises the steps of: (a) obtaining a probe having an antibody against the analyte immobilized on the tip of the probe, wherein the diameter of the tip surface is ≤5 mm; (b) dipping the probe in a baseline vessel comprising an aqueous solution having pH of 6.0-8.5 for a first period of time to determine a baseline interferometry pattern of the probe tip; (c) dipping the probe tip into a sample vessel containing a liquid sample having the analyte for a second period of time to determine a second interferometry pattern of the immunocomplex formed at the probe tip; (d) determining the analyte concentration in the sample by measuring the interferometry phase shift between the second interferometry pattern and the baseline interferometry pattern, and quantitating the phase shift against a calibration curve; (e) dipping the probe tip in an acidic solution having pH about 1.0-4.0 to elute the immunocomplex from the probe tip; and (f) repeating steps (b)-(e) with a second liquid sample in a second sample vessel in a second cycle, whereby the analyte in multiple liquid samples is detected. The method uses the same probe, the same reagent solution, and the same washing solution in all cycles of reaction. The assay protocol of this first embodiment is illustrated in FIG. 4.

In step (a) of the present method, a probe having a small tip for binding an analyte is obtained. The tip has a smaller surface area with a diameter ≤5 mm, preferably ≤2 mm or ≤1 mm. The small surface of the probe tip endows it with several advantages. In a solid phase immunoassays, having a small surface area is advantageous because it has less non-specific binding and thus produces a lower background signal. Further, the reagent or sample carry over on the probe tip is extremely small due to the small surface area of the tip. This feature makes the probe tip easy to wash, and causes negligible contamination in the wash solution since the wash solution has a larger volume. Another aspect of the small surface area of the probe tip is that it has small binding capacity. Consequently, when the probe tip is immersed in a reagent solution, the binding of the reagent does not consume a significant amount of the reagent. The reagent concentration is effectively unchanged. Negligible contamination of the wash solution and small consumption of the reagents enable the reagents and the wash solution to be re-used many times, for example, 3-20 times.

In step (b), the probe is dipped in a baseline vessel (a pre-read vessel) comprising an aqueous solution having pH of 6.0-8.5 for a first period of time (e.g., 5 seconds to 5 minutes, 10 seconds to 2 minutes, or 30 seconds to 1 minute), to determine a baseline interferometry pattern of the probe tip. The baseline vessel (or pre-read vessel) contains an aqueous solution such as water or a buffer having pH between 6.0 to 8.5. Preferably, the aqueous solution contains 1-10 mM or 1-100 mM of phosphate buffer, tris buffer, citrate buffer or other buffer suitable for pH between 6.0-8.5, to neutralize the probe after low pH regeneration. Pre-read is necessary before the first sample binding to establish a baseline of for the first cycle reaction. Pre-read is also necessary after the regeneration of the probe tip and before the next sample binding to establish a baseline for subsequent cycles. After each cycle after regeneration of probe by low pH, the pre-read baseline interference pattern can be the same as, or different from the pre-read baseline interference pattern of the previous cycle, due to the change of the binding property of the immobilized capture antibody caused by the denaturing condition.

In step (c) of the method, the probe tip is dipped into a sample vessel (or a sample chamber or a sample well) containing a liquid sample having the analyte for a second period of time (e.g., 5 seconds to 5 minutes, 10 seconds to 2 minutes, or 30 seconds to 1 minute), to determine a second interferometry pattern of the immunocomplex formed at the probe tip.

In step (d), the analyte concentration in the sample is quantitated by determining the interferometry phase shift between the second interferometry pattern and the baseline interferometry pattern, and quantitating the wavelength phase shift against a calibration curve to determine the analyte concentration. The phase shift can be monitored either kinetically or determined by the difference between starting time point (T0) and end time point (T1).

The calibration curve is typically pre-established before assaying the samples according to the methods known to a person skilled in the art. In one embodiment, the interference pattern of the same sample remains constant at each cycle, and the calibration curves are the same for each cycle. In another embodiment, the interference pattern of the same sample changes at each cycle, and a cycle-specific calibration curve needs to be established for each cycle. In these instances with changes in interference pattern, samples are quantitated against a cycle-specific calibration curve, and the quantitated results are shown to be consistent in spite of the change of interference pattern at different cycles.

After step (d), the probe is optionally washed 1-5 times, preferably 1-3 times in a wash vessel containing a wash solution. The wash solution typically contains a buffer and optionally a surfactant such as Tween 20. This washing step may not be required because the amount of the carried-over solution is minimal due to a small binding surface area.

In step (e), the probe is dipped in a vessel containing a low pH buffer to regenerate the probe. The probe is regenerated by employing a denaturing condition that dissociates the immune complexes bound to the capture antibody on a solid phase, but does not denature or dissociate the capture antibody from the solid phase to a degree that affects the assay performance. In general, an acid or an acidic buffer having pH about 1 to about 4 is effective to regenerate the antibody probe of the present invention. For example, hydrochloric acid, sulfuric acid, nitric acid, acetic acid can be used to regenerate the probe. The regeneration procedures can be one single acidic treatment, followed by neutralization. For example, a single pH 1-3, or pH 1.5-2.5 (e.g., pH 2) exposure ranging from 10 seconds to 2 minutes is effective. The regeneration procedures can also be a “pulse” regeneration step, where the probe is exposed to 2-5 cycles (e.g. 3 cycles) of a short pH treatment (e.g., 10-20 seconds), followed by neutralization at pH 6.5-8.0 (e.g., 10-20 seconds).

After regeneration of the probe, steps of (b)-(e) are repeated with a different sample in a subsequent cycle, for 1-10, 1-20, 1-25, 3-20, 5-20, 5-25, or 5-30 times, with the same probe and the same reagents.

When step (b) is repeated, the low pH treated probe is conveniently neutralized in the pre-read baseline vessel of step (b).

### Capture Antibody

The inventor has discovered that for certain antibodies such as mouse anti-human CRP monoclonal antibody CRP 30 (an IgG1 isotype) from Hytest (Turku, Finland), when used as a capture antibody in the present method, the interference pattern after each cycle of reaction and regeneration remains constant for at least 10 cycles using the same probe and the same reagents. Because the capture antibody anti-CRP antibody CRP 30 provides a consistent interference pattern through multiple regeneration cycles, such effect enables sample quantification with a single calibration curve, and thus provides convenience and high precision.

The inventor has discovered that for some antibodies, such as anti-human CRP monoclonal antibody C7 from HyTest, when used as capture antibodies in the present method, the interference pattern after each cycle of reaction and regeneration changes.

The acid treatment could alter the protein on the surface of the probe to cause the change of the capture antibody binding capacity. In spite of the change of baseline interference pattern at each cycle, consistent quantification of an analyte concentration may be obtained in such case with a cycle specific calibration; i.e., the interference pattern at the completion of each cycle of reaction, after adjusted by the baseline interference pattern, is quantitated against a cycle-specific calibration curve included in the system.

Although cycle-specific calibration curve could resolve the change of interference pattern of some capture antibodies after regeneration of the probe by low pH, it is advantageous to use a capture antibody that does not change the interference pattern after regeneration of the probe by low pH. CRP, like most quantitative immunoassays employed in clinical laboratories, has a defined set of performance parameters that must be met to have clinical utility Minimum detection limit, analytical range, and precision are examples of such performance parameters. With CRP 30 antibody, the assay conditions can be established and remain unchanged during multiple recycles using a single calibration while maintaining its assay performance parameters. Capture antibodies that produce variable signals after regeneration by low pH require cycle specific calibration; in addition, assay parameters are difficult to maintain. Since cycle specific calibration introduces an additional variable, imprecision between cycles is greater. This is a drawback since clinical assays require high precision with coefficient of variation (CV)<10%. Antibodies that lose activity and generate declining wavelength phase shift after low pH treatment typically have a difficulty to maintain precision, minimum detection limit, and analytical range due to decreasing signals

### Detecting an Analyte by a Recycling Protocol—Sandwich Format

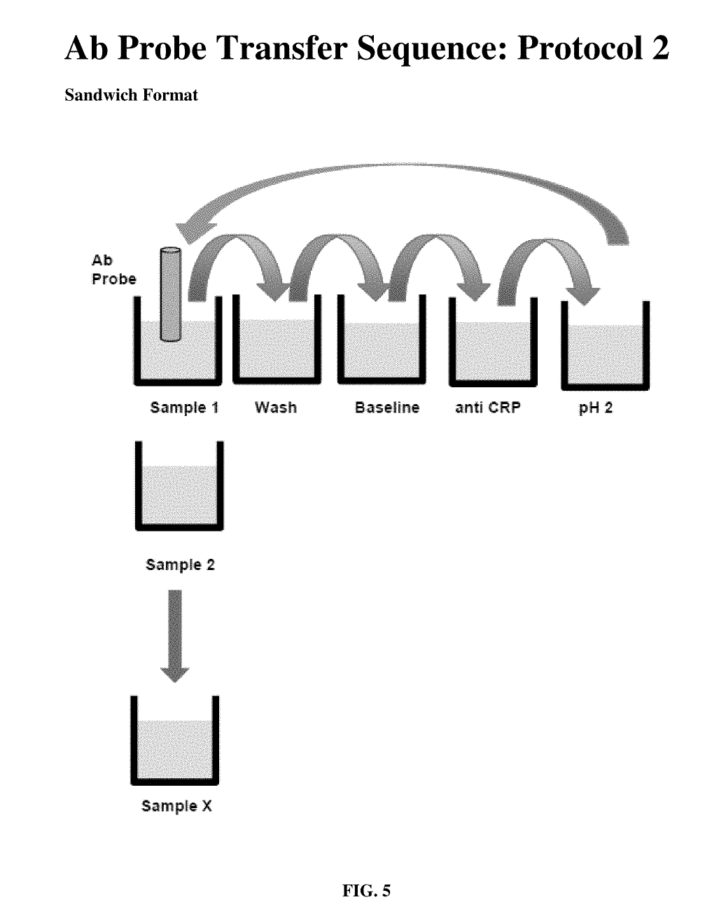

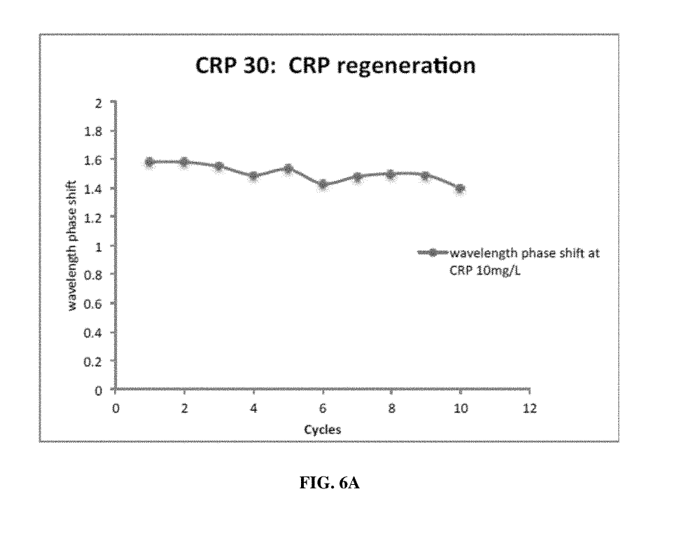

In a second embodiment (sandwich format), a capture antibody is immobilized on the probe, the sample contains an antigen analyte, and the interference pattern shift is determined after the binding by a second antibody. The assay protocol of this second embodiment of the invention is illustrated in FIG. 5.

The sandwich method comprises the steps of: (a) obtaining a probe having a first antibody immobilized on the tip of the probe, wherein the diameter of the tip surface is ≤5 mm; (b) dipping the probe tip into a sample vessel containing a liquid sample having the analyte; (c) dipping the probe tip in a wash vessel containing a buffer to wash the probe; (d) dipping the probe in a baseline vessel comprising an aqueous solution having pH of 6.0-8.5 for a first period of time to determine a baseline interferometry pattern of the probe tip; (e) dipping the probe in a reagent vessel comprising a second antibody having pH of 6.0-8.5 for a second period of time to determine a second interferometry pattern of the immunocomplex formed at the probe tip; (f) determining the analyte concentration in the sample by measuring the interferometry phase shift between the second interferometry pattern and the baseline interferometry pattern, and quantitating the phase shift against a calibration curve; (g) dipping the probe tip in an acidic solution having pH about 1.0-4.0 to elute the immunocomplex from the probe tip; and (h) repeating steps (b)-(g) with a second liquid sample in a second sample vessel in a second cycle, whereby the analyte in multiple liquid samples is detected. The parameters of this second embodiment of the invention are similar or the same as the first embodiment of the invention.

The sandwich format assay protocol has an extra binding step by a second antibody than the first embodiment. However, it has the advantage to resolve non-specific binding caused by non-analyte materials in the sample.

In an interferometry assay, any material that binds to the sensing surface and change the thickness of the biomolecule layer will produce a change in the interferometry signal. Blood samples often contain components such as albumin, rheumatoid factor, heterophile antibodies, and lipids, which are known to bind non-specifically to antibody-coated solid phases. In an interferometry assay, this will generate non-specific signal interfering with the immunoassay. The problem is further challenged by blood varying from sample to sample causing variation in non-specific effects.

The sandwich format protocol of the present invention can minimize non-specific binding effects caused by blood components. In this format, the antibody coated probe is immersed in analyte-containing blood sample, followed by a wash sequence to remove the blood, and then immersed in a second antibody reagent. Washing removes the non-specifically bound materials. The baseline is read after washing, and the binding of the second antibody to the immune complex on the probe in then detected by the interference pattern.

### Unitized Immunoassay Strips

The present invention is further directed to a cartridge (a strip) for an immunoassay test. This unitized cartridge can be used for 2-20, or 3-20 cycles to measure 2-20, or 3-20 different samples. The cartridge comprises (a) a probe well comprising a probe, wherein the probe has a bottom tip coated with a first antibody, (b) a baseline well (or a pre-read well) to establish the baseline interference pattern, (c) a sample well to receive a sample, (d) a low pH well to provide pH of 1-4, (e) optionally a reagent well; and (f) one or more wash wells each containing a wash solution.

A sample well is a well that receives a sample containing an analyte. A sample well can be a blank well, or it can contain detergents, blocking agents and various additives for the immunoassay, either in a dry format or in a wet (liquid) format.

A reagent well contains reagents such as an antibody that reacts with the analyte to form an immunocomplex. The reagents can be in a wet format or in a dry format. The wet format contains a reagent in an assay buffer. The wet format is typically in a small liquid volume (<10 μL, e.g., 5 μL). An assay buffer typically includes a buffer (e.g., phosphate, tris), a carrier protein (e.g., bovine serum albumin, porcine serum albumin, and human serum albumin, 0.1-50 mg/mL), a salt (e.g., saline), and a detergent (e.g., Tween, Triton). An example of an assay buffer is phosphate buffered saline, pH 7.4, 5 mg/ml bovine serum albumin, 0.05% Tween 20. The assay buffer optionally contains a blocking agent in an amount of 1-500 μg/mL. The final formulation will vary depending on the requirements of each analyte assay. The dry format is the dry form of the reagent in an assay buffer. The dry format includes lyophilization cake, powder, tablet or other formats typical in diagnostic kits. The dry format is to be reconstituted to a wet format by a reconstitution buffer or a wash buffer.

The cartridge optionally comprises one or more washing wells each containing an aqueous solution. The wash wells contain a wash buffer to wash the probe after binding steps in the sample well and reagent well. One to four wash wells (e.g., 1, 2, 3, or 4 wells) are dedicated for washing after each binding step. Wash buffers contain detergents. Any detergent typically used in immunoassays (e.g., Tween, Triton) can be used in this invention.

The openings of the reagent well and wash well(s) are sealed with a foil or a film. The seal is penetrable. The wells may be opened by piercing the seal by a manual or automated device.

The invention is illustrated further by the following examples that are not to be construed as limiting the invention in scope to the specific procedures described in them.

### EXAMPLES

### Example 1. Preparing Streptavidin-Coated Probes

### Preparing Aminopropropylsilane-Coated Probe

A glass rod (a monolithic substrate), 1 mm diameter and 2 cm in length, had both coupling end and sensing end polished. The sensing end was first coated with a SiO₂ coating layer (a thin-film layer) with a thickness of 650 nm using a physical vapor deposition technology, and then deposited with aminopropylsilane (APS) using a chemical vapor deposition process (Yield Engineering Systems, 1224P) following manufacturer's protocol. APS is deposited to enable protein immobilization. APS adsorbs protein to the surface of the probe by a combination of hydrophobic and ionic interaction. APS is only a monolayer, about 7 nm thick.

### Preparing Cross-Linked Streptavidin

Cross-linked streptavidin (SA) was prepared in the following manner 10 mg of Streptavidin monomer (Scripps Labs) dissolved at 10 mg/ml in 100 mM sodium phosphate buffer containing 150 mM NaCl, pH 7.2 (PBS) was derivatized with a 10M excess of the bifunctional reagent, N-succinimidyl-A-acetylthioacetate (SATA), dissolved at 40 mg/ml in dimethyl formamide, (DMF) for 2 hours. At the same time 10 mg of Streptavidin dissolved at 10 mg/ml in PBS was derivatized with a 10 M excess of the bifunctional reagent, sulfosuccinimidyl 4-(N-maleimidomethyl) cyclohexane-1-carboxylate (Sulfo-SMCC), dissolved at 40 mg/ml in water for 1 hour. The samples were purified to remove excess bifunctional reagents using 10 mL crosslinked dextran desalting columns. The purified samples were combined in equal molar ratios and degassed under vacuum. The combined volume was determined and then a quantity of 1 M hydroxylamine hydrochloride, previously degassed under vacuum, was added so that the final concentration of the Hydroxylamine Hydrochloride in the sample was 50 mM. The addition of hydroxylamine hydrochloride deblocks the acetylated sulfhydryl group on the SATA modified protein creating a free sulfhydryl group to react with the maleimide groups on the Sulfo-SMCC modified proteins. The reaction was carried out for 1 hour and 30 minutes at room temperature. Excess maleimide groups were capped with β-mercaptoethanol for 15 minutes. Excess sulfhydryl groups were capped with N-ethylmaleimide for 15 minutes. Then the sample was dialyzed against 5 L of PBS. The cross-linked SA is then purified in a S-300 column

### Preparing Streptavidin-Coated Probe

The APS probe was dipped into cross-linked SA (100 μg/mL in PBS) under orbital shaking (1000 rpm) for 10 minutes; then the probe was washed in PBS for 15 seconds three times. The probe was then dipped into a 15% sucrose solution, followed by drying in a convention oven at 37 C for 30 minutes.

### Example 2. Preparing Anti-CRP Antibody Coated Probe

Anti-CRP antibodies were biotinylated by a standard method.

The streptavidin-coated probe of Example 1 was immersed in a 10 μg/ml solution of biotinylated anti-CRP (Hytest CRP 30) or biotinylated anti-CRP (Hytest C7) for 10 minutes, followed by immersion in 10% sucrose for 30 seconds then dried at 30° C. for one hour and then stored in a dry condition.

### Example 3: CRP Assay, Protocol 1 (FIG. 4)

1.) Anti-CRP coated probe is immersed in buffer for 30 seconds to establish baseline interferometry phase shift signal.
2.) Probe is immersed in CRP samples and interferometry is measured at 60 sec for interferometry phase shift signal from T0.
3.) Wash probe in buffer 10 seconds (optional)
4) Immerse probe in pH 2 buffer for 15 seconds
5.) Repeat steps 1-4 with probe, use new CRP sample in step 2, same reagents for steps 1, 3 & 4. Recycle probe and reagents can be done up 20 times.

### Example 4. CRP Assay, Protocol 1, Comparing CRP 30 and C7 as Capture Anti-CRP Antibodies

Buffer (PBS) samples containing 10 mg/L CRP were tested for wavelength phase shift for 10 cycles following the procedures of Example 3, using two different anti-CRP coated probes (CRP 30 or C7).

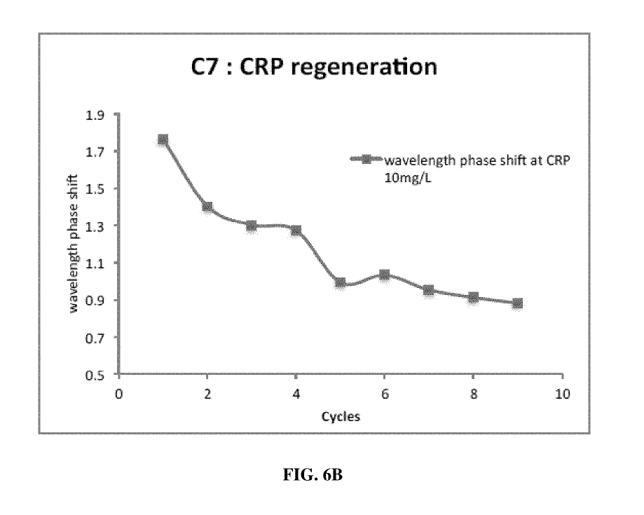

FIG. 6A shows the wavelength phase shifts (nm) from Cycle 1 to Cycle 10 when anti-CRP antibody CRP 30 was used as a capture antibody. The results show consistent wavelength phase shifts from Cycle 1 to Cycle 10.

FIG. 6B shows the wavelength phase shifts (nm) from Cycle 1 to Cycle 9 when anti-CRP antibody C7 was used as a capture antibody. The results show that wavelength phase shifts dropped significantly from Cycle 1 to Cycle 9.

### Example 5: CRP Assay, Sandwich Format, Using Anti-CRP CRP 30 as a Capture Antibody and Anti-CRP C5 as a Signal Antibody

1.) Pre-wash anti-CRP coated probe in PBS, pH 7.4 for 10 seconds
2) Immerse anti-CRP coated probe in sample containing 20 mg/L CRP and incubate for 3 minutes at 500 rpm
3.) Wash probe in PBS, 3 times, 10 seconds, 500 rpm
4) Immerse probe in buffer for 30 seconds to read a baseline phase shift interference signal
5.) Transfer probe to anti-CRP C5 antibody (20 mg/L), 500 rpm, and monitor phase shift interference signal for 3 minutes, and read the wavelength phase shift (nm) at 3 minutes from T0.
6.) Three washes in PBS, 10 seconds, 500 rpm
7) Immerse probe in regeneration buffer (10 mM glycine, pH 2), 10 seconds, 500 rpm
8.) Wash probe in PBS, 10 seconds, 500 rpm
9.) Repeat 6 and 7 twice
10.) Return to 2, for subsequent CRP cycle

The entire procedures were conducted at room temperature.

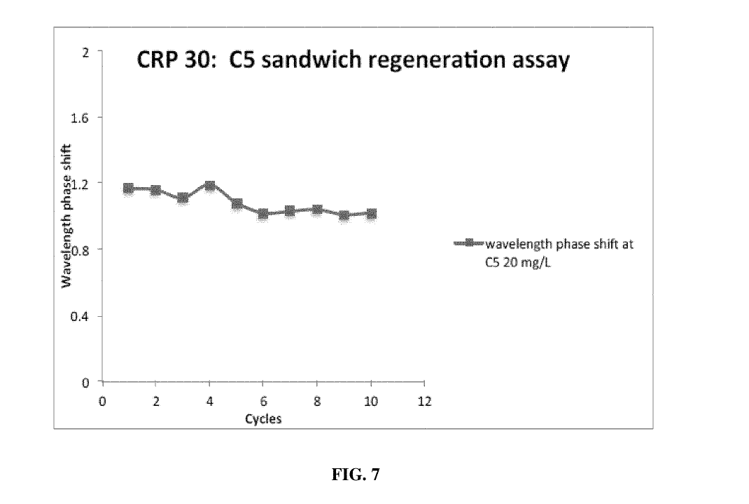

FIG. 7 shows the results of wavelength phase shifts (nm) from Cycle 1 to Cycle 10 when anti-CRP antibody CRP 30 was used as a capture antibody, and anti-CRP antibody C5 was used as a signal antibody. The results show consistent wavelength phase shifts from Cycle 1 to Cycle 10.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of detecting an analyte in multiple liquid samples, comprising the steps of:
(a) obtaining a probe having an antibody against the analyte immobilized on the tip of the probe, wherein the diameter of the tip surface is ≤5 mm;
(b) dipping the probe in a baseline vessel comprising an aqueous solution having pH of 6.0-8.5 for a first period of time to determine a baseline interferometry pattern of the probe tip;
(c) dipping the probe tip into a sample vessel containing a liquid sample having the analyte for a second period of time to determine a second interferometry pattern of the immunocomplex formed at the probe tip;
(d) determining the analyte concentration in the sample by measuring the interferometry phase shift between the second interferometry pattern and the baseline interferometry pattern, and quantitating the phase shift against a calibration curve;
(e) dipping the probe tip in an acidic solution having pH about 1.0-4.0 to elute the immunocomplex from the probe tip; and
(f) repeating steps (b)-(e) with a second liquid sample in a second sample vessel in a second cycle, whereby the analyte in multiple liquid samples is detected.

2. The method of claim 1, wherein the calibration curves in step (d) are the same for all cycles of quantitation.

3. The method of claim 2, wherein the analyte is C-reactive protein (CRP), and the antibody is mouse anti-human CRP monoclonal antibody CRP 30.

4. The method of claim 1, wherein the acidic solution in step (e) has a pH of 1.5-2.5.

5. The method of claim 1, where in step (e), the probe tip is exposed to the acidic solution one time for 10 second to 2 minutes.

6. The method of claim 1, where in step (e), the probe tip is exposed to a pulse treatment of 2-5 cycles of the acidic solution treatment followed by neutralization in the read vessel for 10-20 seconds.

7. The method of claim 1, wherein steps (b)-(e) are repeated 3-20 times.

8. A method of detecting an analyte in multiple liquid samples, comprising the steps of:
(a) obtaining a probe having a first antibody immobilized on the tip of the probe, wherein the diameter of the tip surface is ≤5 mm;
(b) dipping the probe tip into a sample vessel containing a liquid sample having the analyte;
(c) dipping the probe tip in a wash vessel containing a buffer to wash the probe;
(d) dipping the probe in a baseline vessel comprising an aqueous solution having pH of 6.0-8.5 for a first period of time to determine a baseline interferometry pattern of the probe tip;
(e) dipping the probe in a reagent vessel comprising a second antibody having pH of 6.0-8.5 for a second period of time to determine a second interferometry pattern of the immunocomplex formed at the probe tip;
(f) determining the analyte concentration in the sample by measuring the interferometry phase shift between the second interferometry pattern and the baseline interferometry pattern, and quantitating the phase shift against a calibration curve;
(g) dipping the probe tip in an acidic solution having pH about 1.0-4.0 to elute the immunocomplex from the probe tip; and
(h) repeating steps (b)-(g) with a second liquid sample in a second sample vessel in a second cycle, whereby the analyte in multiple liquid samples is detected.

9. The method of claim 8, wherein the calibration curves in step (f) are the same for all cycles of quantitation.

10. The method of claim 9, wherein the analyte is CRP, and the first antibody is mouse anti-human CRP monoclonal antibody CRP 30.

11. The method of claim 8, wherein the acidic solution in step (g) has a pH of 1.5-2.5.

12. The method of claim 8, where in step (g), the probe tip is exposed to the acidic solution one time for 10 second to 2 minutes.

13. The method of claim 8, where in step (g), the probe tip is exposed to a pulse treatment of 2-5 cycles of the acidic solution treatment followed by neutralization in the read vessel for 10-20 seconds.

14. The method of claim 8, wherein steps (b)-(g) are repeated 3-20 times.
